(19) United States
(12) Patent Application Publication
(10) Pub. No.: US 2025/0339130 A1
(43) Pub. Date: Nov. 6, 2025

(54) ACUTE KIDNEY INJURY RISK ESTIMATOR

(71) Applicant: Becton, Dickinson and Company, Franklin Lakes, NJ (US)

(72) Inventor: Blake W. Axelrod, South Pasadena, CA (US)

(21) Appl. No.: 19/265,985

(22) Filed: Jul. 10, 2025

Related U.S. Application Data
(63) Continuation of application No. PCT/US2024/011069, filed on Jan. 10, 2024.
(60) Provisional application No. 63/479,250, filed on Jan. 10, 2023.

(51) Int. Cl. A61B 8/00; A61B 8/06; A61B 90/00

(52) U.S. Cl. CPC A61B 8/5223; A61B 8/06; A61B 8/4236; A61B 8/4488; A61B 8/4494; A61B 8/461; A61B 8/488; A61B 90/06; A61B 2090/064

(57) ABSTRACT

A system includes an ultrasound transducer probe with a two-dimensional array of transducer elements that is configured to measure a Doppler flow signal of a targeted organ blood flow of a patient. An adhesive patch is connected to the ultrasound transducer probe and is configured to attach the ultrasound transducer probe to the patient. The system includes a blood flow monitor in communication with the ultrasound transducer probe. The blood flow monitor includes a processor and system memory that stores monitoring software code. The processor is configured to execute the monitoring software code to determine at least one characteristic associated with the targeted organ blood flow of the patient. The processor is also configured to execute the monitoring software code to monitor over time the at least one characteristic associated with the targeted organ blood flow of the patient during a surgery, medical procedure, or medical observation of the patient.

### CROSS-REFERENCE TO RELATED APPLICATION(S)

This application is a continuation of International Application No. PCT/US2024/011069, filed Jan. 10, 2024, and entitled “ACUTE KIDNEY INJURY RISK ESTIMATOR,” which claims the benefit of U.S. Provisional Application No. 63/479,250, filed Jan. 10, 2023, and entitled “ACUTE KIDNEY INJURY RISK ESTIMATOR,” the disclosures of which are hereby incorporated by reference in their entireties.

### BACKGROUND

Acute kidney injury (AKI) occurs when a kidney experiences a sudden decrease in function. AKI can be a complication from major abdominal surgery and may increase a risk of chronic kidney disease in a patient if AKI is not detected and treated at an early stage. Decreased perfusion to the kidney(s) during surgery is one cause of AKI. Detecting AKI in a patient is traditionally done by viewing two biomarkers in the patient. The first biomarker is analyzing urine output of the patient and the second biomarker is measuring serum creatinine from a blood sample of the patient. These biomarkers generally do not show up in the patient until about eight hours to forty-eight hours after the injury has occurred to the kidney(s). Due to the late onset of these biomarkers, physicians can only use these biomarkers to detect whether AKI has occurred a relatively long time after the kidney has been damaged, and cannot use these biomarkers to monitor health of the kidneys in real time during a surgery. The ability to monitor the health of the kidneys during surgery would not only allow physicians the ability of early detection of AKI, but possibly the ability to prevent AKI in the patient.

### SUMMARY

A method is disclosed for monitoring a patient during a surgery, a medical procedure, or a medical observation with a blood flow monitor in communication with an ultrasound transducer probe. The method includes the step of obtaining a Doppler flow signal of a targeted organ blood flow of the patient with the ultrasound transducer probe. The ultrasound transducer probe is attached in a stationary position to an abdomen of the patient. A processor of the blood flow monitor determines at least one characteristic associated with the targeted organ blood flow of the patient from the Doppler flow signal. The processor also determines a baseline value of the at least one characteristic. The processor continuously monitors over time the Doppler flow signal and the at least one characteristic during the surgery, medical procedure, or medical observation of the patient.

A system includes an ultrasound transducer probe with a two-dimensional array of transducer elements that is configured to measure a Doppler flow signal of a targeted organ blood flow of a patient. An adhesive patch is connected to the ultrasound transducer probe. The adhesive patch is configured to attach the ultrasound transducer probe to the patient and maintain contact between the patient and the ultrasound transducer probe without an operator. The system also includes a blood flow monitor in communication with the ultrasound transducer probe. The blood flow monitor includes a processor and system memory that stores monitoring software code. The processor is configured to execute the monitoring software code to determine at least one characteristic associated with the targeted organ blood flow of the patient. The processor is also configured to execute the monitoring software code to monitor over time the at least one characteristic associated with the targeted organ blood flow of the patient during a surgery, medical procedure, or medical observation of the patient.

### BRIEF DESCRIPTION OF THE DRAWINGS

FIG. 1 is a schematic diagram illustrating an example monitoring system with a blood flow monitor and an ultrasound transducer probe attached to an abdomen of a patient by an adhesive patch.

FIG. 2 is another schematic diagram illustrating the blood flow monitor of FIG. 1.

FIG. 3 is a schematic diagram of an ultrasound transducer probe attached to an abdomen of a patient by an adhesive patch to monitor a kidney of the patient.

FIG. 4A is another schematic diagram of an ultrasound transducer probe attached to an abdomen of a patient by an adhesive patch to monitor a kidney of the patient.

FIG. 4B is another schematic diagram of an ultrasound transducer probe attached to an abdomen of a patient by an adhesive patch to monitor a kidney of the patient.

FIG. 5 is a block diagram of a method for continuously monitoring an organ of a patient during a surgery, medical procedure, or medical observation with a blood flow monitor for risk of organ injury.

FIG. 6 is a block diagram of a method for continuously monitoring a kidney of a patient during a surgery, medical procedure, or medical observation with a blood flow monitor for risk of acute kidney injury.

FIG. 7 is a block diagram of a method for continuously monitoring a kidney of a patient during a surgery, medical procedure, or medical observation with a blood flow monitor for risk of acute kidney injury.

FIG. 8 is a block diagram of a method for continuously monitoring a kidney of a patient during a surgery, medical procedure, or medical observation with a blood flow monitor for risk of acute kidney injury.

FIG. 9 is a schematic diagram illustrating an example monitoring system with a blood flow monitor, an ultrasound transducer probe attached to an abdomen of a patient by an adhesive patch, and a pressure sensor connected to the patient for sensing hemodynamic data representative of an arterial pressure of the patient.

### DETAILED DESCRIPTION

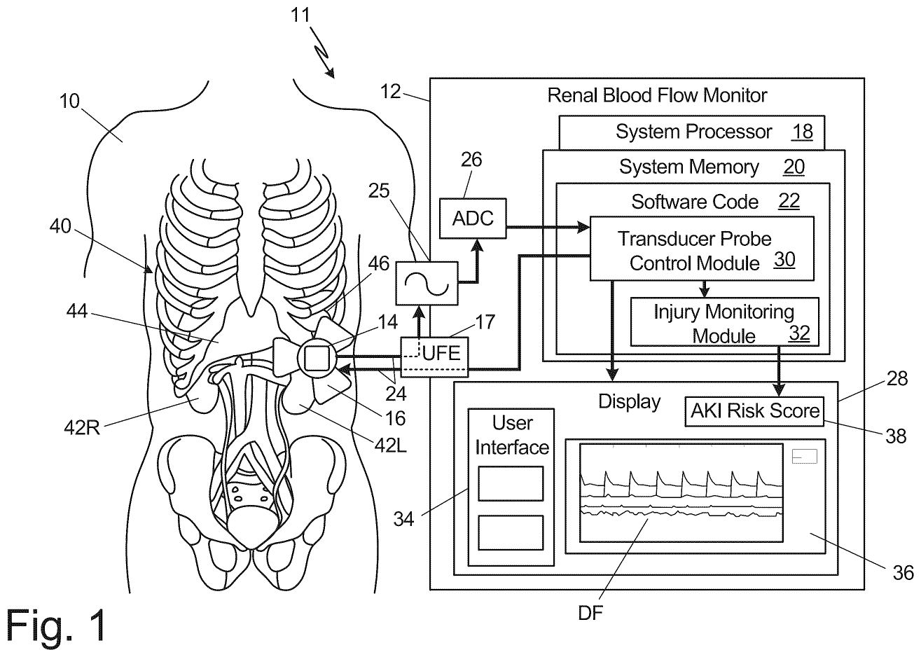

The present disclosure is directed to a system and a method to monitor in real time a blood flow of an abdominal organ, such as a kidney, of a patient during a surgery, medical procedure, or medical observation. The system includes a blood flow monitor with an ultrasound transducer probe. The system also includes an adhesive patch that can attach the ultrasound transducer probe to a patient and keep the ultrasound transducer probe attached to the patient through the surgery, medical procedure, or medical observation of the patient without assistance from an ultrasound operator. The blood flow monitor also includes an algorithm for continuously determining an injury risk score of the abdominal organ. The injury risk score can be continuously updated and outputted to a display during the surgery, medical procedure, or medical observation so that medical personnel can be informed in real time of the risk of injury to the abdominal organ. The blood flow monitoring system is described in detail below with reference to FIGS. 1-8.

FIG. 1 is a schematic diagram of patient 10 and monitoring system 11 that continuously monitors an organ blood flow of patient 10 during a surgery, medical procedure, or medical observation. As shown in the example of FIG. 1, monitoring system 11 can include renal blood flow monitor 12, ultrasound transducer probe 14, adhesive patch 16, ultrasound front-end circuitry 17, system processor 18, system memory 20 with software code 22, probe cables 24, analog-to-digital (ADC) converter 26, and display 28. Software code 22 can include transducer probe control module 30 and injury monitoring module 32. Display 28 can include user interface 34, plot 36, and injury score indicator 38. FIG. 1 also shows abdomen 40 of patient 10 along with kidneys 42L and 42R, liver 44, and spleen 46. In the example of FIG. 1, monitoring system 11 is monitoring a renal blood flow of kidney 42L of patient 10. In other examples, monitoring system 11 can be used to monitor hepatic blood flow of liver 44, to monitor celiac blood flow of spleen 46, the pancreas (not shown), and the stomach (not shown) of patient 10, and/or to monitor portal blood flow from the stomach of patient 10. Thus, renal blood flow monitor 12 can be adapted as an organ blood flow monitor 12 for any abdominal organ of patient 10.

Renal blood flow monitor 12, can be, e.g., an integrated hardware unit that includes system processor 18, system memory 20, display 28, ultrasound front-end circuitry 17, and ADC 26. In other examples, any one or more components and/or described functionality of organ blood flow monitor can be distributed among multiple hardware units. For instance, in some examples, display 28 can be a separate display device that is remote from and operatively coupled with renal blood flow monitor 12. In general, though illustrated and described in the example of FIG. 1 as an integrated hardware unit, it should be understood that renal blood flow monitor 12 can include any combination of devices and components that are electrically, communicatively, or otherwise operatively connected to perform functionality attributed herein to renal blood flow monitor 12.

Ultrasound transducer probe 14 can be attached or secured to patient 10 by adhesive patch 16. In the example of FIG. 1, ultrasound transducer probe 14 is positioned on abdomen 40 of patient 10 over at least a portion of kidney 42L. Adhesive patch 16 can include a sheet of structural material, such as fabric or flexible plastic, with a layer of bonding adhesive deposited on a face of the sheet. Adhesive patch 16 can be bonded to or mechanically connected to ultrasound transducer probe 14, or to a frame (not shown) connected to a base of ultrasound transducer probe 14, and can extend outward from ultrasound transducer probe 14 along a surface of abdomen 40 of patient 10. In other examples, adhesive patch 16 can be placed over ultrasound transducer probe 14 to attach ultrasound transducer probe 14 to abdomen 40 of patient 10. Adhesive patch 16 keeps ultrasound transducer probe 14 attached to patient 10 and secured in place throughout a duration of the surgery, medical procedure, or medical observation of patient 10. Since adhesive patch 16 keeps ultrasound transducer probe 14 immobile and in contact with patient 10, an ultrasound operator or technician is not needed during the surgery, medical procedure, or medical observation to keep ultrasound transducer probe 14 in position. A coupling layer (not shown) with a couplant material can be positioned between a skin of patient 10 and ultrasound transducer probe 14. The coupling layer enables ultrasonic energy transmission between the skin of patient 10 and ultrasound transducer probe 14.

In the example of FIG. 1, the ultrasound transducer probe 14 detects and senses a Doppler flow signal DF of the renal blood flow of kidney 42L. Ultrasound transducer probe 14 can be operatively connected to renal blood flow monitor 12 by cables 24. Via cables 24, ultrasound transducer probe 14 can receive electrical signals from the ultrasound front-end circuitry 17 of the renal blood flow monitor 12 and can relay the received ultrasound signals from patient 10 to renal blood flow monitor 12 for extraction of the Doppler flow signal DF of the renal blood flow of kidney 42L. In other examples, ultrasound front-end circuitry 17 is combined with ultrasound transducer probe 14, can be battery powered and can include a receiver to wirelessly receive commands from renal blood flow monitor 12. The combined ultrasound front-end circuitry 17 and ultrasound transducer probe 14 can also include a transmitter to wirelessly communicate the Doppler flow signal DF of the renal blood flow of kidney 42L to renal blood flow monitor 12 for analysis. In some examples, the combined ultrasound transducer probe 14 and ultrasound front-end circuitry 17 provide the Doppler flow signal DF to renal blood flow monitor 12 as analog signal 25, which is converted by ADC 26 to digital hemodynamic data representative of the renal blood flow of kidney 42L. In other examples, the combined ultrasound transducer probe 14 and ultrasound front-end circuitry 17 can provide the sensed Doppler flow signal DF to renal blood flow monitor 12 in digital form, in which case renal blood flow monitor 12 may not include or utilize ADC 26. In yet other examples, ultrasound transducer probe 14 can provide the Doppler flow signal DF of the renal blood flow of kidney 42L to blood flow monitor 12 as analog signal 25, which is analyzed in its analog form by blood flow monitor 12.

System memory 20 can be configured to store information within renal blood flow monitor 12 during operation. System memory 20, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 20 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

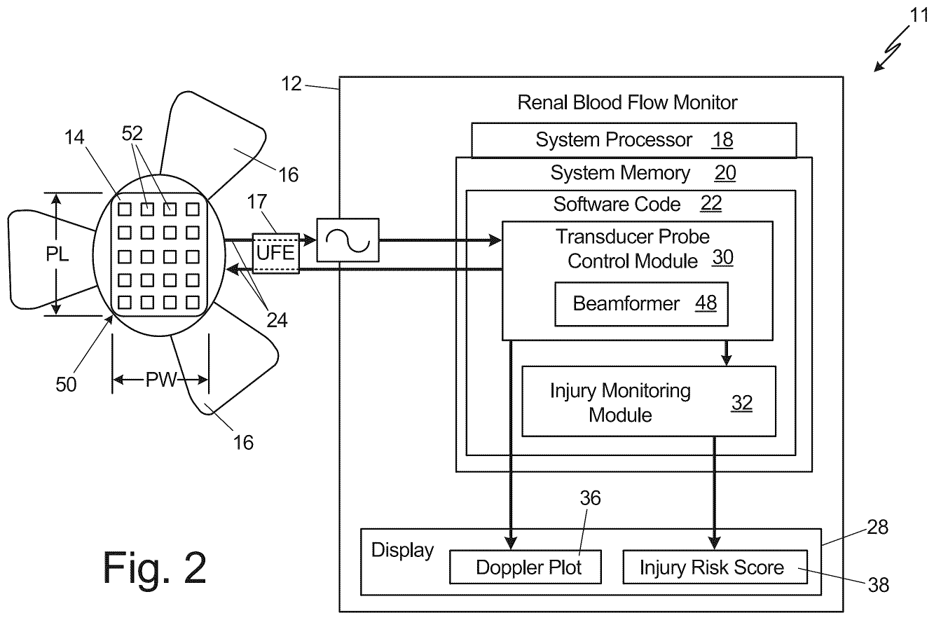

As shown in FIG. 1, system memory 20 of renal blood flow monitor 12 can store software code 22 which forms a monitoring model of renal blood flow monitor 12. Software code 22 can include transducer probe control module 30 for controlling and commanding ultrasound transducer probe 14. Transducer probe control module 30, as discussed in greater detail below with reference to FIG. 2, includes a beamformer that keeps ultrasound transducer probe 14 aimed at the renal blood flow of kidney 42L so that ultrasound transducer probe 14 continuously senses and communicates the Doppler flow signal DF of the renal blood flow to renal blood flow monitor 12 throughout the surgery, medical procedure, or medical observation of patient 10. Software code 22 can also include injury monitoring module 32 which includes acute kidney injury (AKI) monitoring software code and/or specific organ injury (SOI) monitoring software code. This code is monitoring software code that allows injury monitoring module 32 to determine, in real time, a characteristic of the renal blood flow of patient 10, monitor the characteristic of the renal blood flow over time, and determine an AKI risk score of patient 10 from the characteristic and the Doppler flow signal DF of the renal blood flow of kidney 42L. The AKI risk score represents the probability that kidney 42L is experiencing or approaching an AKI. When monitoring system 11 is used to monitor an organ other than kidneys 42L and 42R of patient 10, injury monitoring module 32 can be adapted to determine a real-time organ injury risk score from the Doppler flow signal of the organ blood flow of the organ that is being monitored, such as liver 44.

System processor 18 is a hardware processor configured to execute software code 22, which implements transducer probe control module 30 and injury monitoring module 32, to continuously sense the Doppler flow signal DF and monitor the Doppler flow signal for AKI of kidney 42L. Examples of system processor 18 can include any one or more of a microprocessor, a controller, a digital signal processor (DSP), an application specific integrated circuit (ASIC), a field-programmable gate array (FPGA), or other equivalent discrete or integrated logic circuitry.

Display 28 provides user interface 34, which includes control elements that enable user interaction with renal blood flow monitor 12 and/or other components of monitoring system 11. Display 28 is in communication with system processor 18 and is configured to provide plot 36 in real time of the Doppler flow signal DF of the renal blood flow of kidney 42L. In addition to showing plot 36 of Doppler flow signal DF, display 28 can also provide an audible representation of Doppler flow signal DF via a speaker. Display 28, as shown in FIG. 1, also shows an injury score indicator 38, which is a representation of the real-time AKI risk score of patient 10 determined from the Doppler flow signal DF by system processor 18 and injury monitoring module 32. Display 28 can also include a sensory alarm to alert medical personnel when the real-time AKI risk score of patient 10 is approaching or exceeding a predetermined threshold. The sensory alarm can be implemented as one or more of a visual alarm, an audible alarm, a haptic alarm, or other type of sensory alarm. For instance, the sensory alarm can be invoked as any combination of flashing and/or colored graphics shown by user interface 34 on display 28, a warning sound such as a siren or repeated tone, and a haptic alarm configured to cause renal blood flow monitor 12 to vibrate or otherwise deliver a physical impulse perceptible to medical personnel.

Display 28 can be a liquid crystal display (LCD), a light-emitting diode (LED) display, an organic light-emitting diode (OLED) display, or other display device suitable for providing information to users in graphical form. User interface 34 can include graphical and/or physical control elements that enable user input to interact with renal blood flow monitor 12 and/or other components of monitoring system 11. In some examples, user interface 34 can take the form of a graphical user interface (GUI) that presents graphical control elements presented at, e.g., a touch-sensitive and/or presence sensitive display screen of display 28. In such examples, user input can be received in the form of gesture input, such as touch gestures, scroll gestures, zoom gestures, or other gesture input. In certain examples, user interface 34 can take the form of and/or include physical control elements, such as a physical buttons, keys, knobs, or other physical control elements configured to receive user input to interact with components of monitoring system 11. User interface 34 can include a speaker that allows renal blood flow monitor 12 the ability to generate an audible alarm.

In operation of monitoring system 11, before a surgery, medical procedure, or medical observation begins, a medical worker places ultrasound transducer probe 14 on abdomen 40 of patient 10. The medical worker uses ultrasound transducer probe 14 to locate the Doppler flow signal DF of the renal blood flow of kidney 42L. Ultrasound transducer probe 14 can generate an audible representation of the Doppler flow signal DF to assist the medical worker in locating the Doppler flow signal DF of the renal blood flow of kidney 42L. Once the medical worker finds the Doppler flow signal DF of the renal blood flow of kidney 42L, the medical worker attaches and secures ultrasound transducer probe 14 to patient 10 with adhesive patch 16. Adhesive patch 16 keeps ultrasound transducer probe 14 in constant contact with patient 10 such that ultrasound transducer probe 14 does not shift positions on patient 10 during the surgery, medical procedure, or medical observation and lose the Doppler flow signal DF of the renal blood flow of kidney 42L. Ultrasound transducer probe 14 relays the received ultrasound signals to renal blood flow monitor 12 via cable(s) 24 or wirelessly. In the case of wireless transmission, the ultrasound transducer probe 14 includes the ultrasound front-end circuitry 17. System processor 18 of renal blood flow monitor 12 receives the Doppler flow signal DF and processes the Doppler flow signal DF sequentially or simultaneously through transducer probe control module 30 and injury monitoring module 32.

System processor 18 can execute the AKI monitoring software code of injury monitoring module 32 to establish a baseline value for the renal blood flow of kidney 42L of patient 10 from the Doppler flow signal DF sensed by ultrasound transducer probe 14. Deviations from the baseline value for the renal blood flow can be used as factors by system processor 18 and injury monitoring module 32 to calculate the real-time AKI risk score of kidney 42L. System processor 18 can further execute the AKI monitoring software code of injury monitoring module 32 to continuously monitor the Doppler flow signal DF of the renal blood flow sensed by ultrasound transducer probe 14 throughout a duration of the surgery, medical procedure, or medical observation of patient 10 and estimates the AKI risk score of kidney 42L of patient 10 from the Doppler flow signal DF. System processor 18 outputs the Doppler flow signal DF and the real-time AKI risk score of kidney 42L to display 28. Display 28 produces plot 36 showing the Doppler flow signal DF of the renal blood flow of kidney 42L plotted over time. Display 28 also produces injury score indicator 38 which represents the real-time AKI risk score of kidney 42L in injury score indicator 38.

As the surgery, medical procedure, or medical observation of patient 10 progresses, system processor 18 continues to receive the Doppler flow signal DF from ultrasound transducer probe 14 and continues to output both the Doppler flow signal DF and the real-time AKI risk score of kidney 42L to display 28. If the real-time AKI risk score of kidney 42L changes toward an undesired threshold, or changes at an undesired rate, system processor 18 and display 28 can alert the medical personnel so that the medical personnel can possibly take action to increase kidney perfusion and prevent AKI to kidney 42L, or minimize AKI to kidney 42L. For example, medical personnel can administer medication or fluids that increase the renal blood flow and perfusion to kidney 42L or improves autoregulation of the renal blood flow to kidney 42L. At the end of the surgery, medical procedure, or medical observation, system processor 18 and injury monitoring module 32 can estimate a final AKI risk score for kidney 42L and output the final AKI risk score to display 28. If the final AKI risk score for kidney 42L indicates that kidney 42L has a high risk of AKI, medical personnel can take immediate action to treat kidney 42L without having to wait for biomarkers to appear in blood and urine samples of patient 10. Biomarkers that indicate AKI can take several hours or days to appear in blood and urine samples of patient 10. With monitoring system 11, the medical personnel can determine quickly whether patient 10 needs to be treated for AKI of kidney 42L.

If kidney 42L of patient 10 moves within abdomen 40 of patient 10 during the surgery, medical procedure, or medical observation, transducer probe control module 30 will detect a change in the Doppler flow signal DF and will respond adjusting the focusing location of the set of beams to scan abdomen 40 of patient 10 to relocate the Doppler flow signal DF and aim ultrasound transducer probe 14 at the new location of the Doppler flow signal DF of the renal blood flow of kidney 42L. As discussed below with reference to FIGS. 2-5, renal blood flow monitor 12 can include a beamformer that can steer beam signals produced by an array of transducer elements of ultrasound transducer probe 14.

FIG. 2 is another schematic diagram of renal blood flow monitor 12. As shown in FIG. 2, renal blood flow monitor 12 can include beamformer 48 and ultrasound transducer probe 14 can include array 50 of transducer elements 52. Each transducer element 52 of array 50 can comprise a piezoelectric material, such as lead zirconate titanate, capable of transmitting ultrasound pulses and detecting ultrasound pulses. Array 50 of transducer elements 52 of ultrasound transducer probe 14 can form a two-dimensional phased array with probe length PL and probe width PW. As a phased array, each transducer element 52 in array 50 can pulse individually relative to the other transducer elements 52 in array 50.

In the example of FIG. 2, beamformer 48 drives array 50 of transducer elements 52 via system processor 18 and ultrasound front-end circuitry 17. Beamformer 48 functions as a transducer probe controller with flow signal tracking software code that controls the timing that each transducer element 52 in array 50 emits an ultrasound pulse. Beamformer 48 can time and pattern when each transducer element 52 emits a pulse such that array 50 can form one or more ultrasonic beams and can sweep or steer the one or more ultrasonic beams without physically moving the position of ultrasound transducer probe 14 on patient 10. Beamformer 48 can be a software sub-module of transformer probe control module 30 that can be executed by system processor 18 to control activation of transducer elements 52 of array 50. In other examples, beamformer 48 can be a separate hardware component from system processor 18 and system memory 20 with separate memory and software from software code 22 that coordinates with system processor 18 to control activation of transducer elements 52 of array 50. In the example of FIG. 2, beamformer 48 is housed within renal blood flow monitor 12 as part of transducer probe control module 30 of software code 22 that is executed by system processor 18. In other examples, beamformer 48 can be fully or partially housed within a casing of ultrasound transducer probe 14 as a separate hardware and software unit that coordinates with system processor 18. Housing beamformer 48 in the same unit as renal blood flow monitor 12 (whether as part of software code 22 or as an add-on hardware component) can decrease the overall size and thickness of ultrasound transducer probe 14. Ultrasound transducer probe 14 can be relatively thin and flat in profile, with a thickness that is smaller than a width or diameter of ultrasound transducer probe 14. Attaching ultrasound transducer probe 14 to patient 10 by adhesive patch 30 is easier and more secure when ultrasound transducer probe 14 has a thin and flat profile.

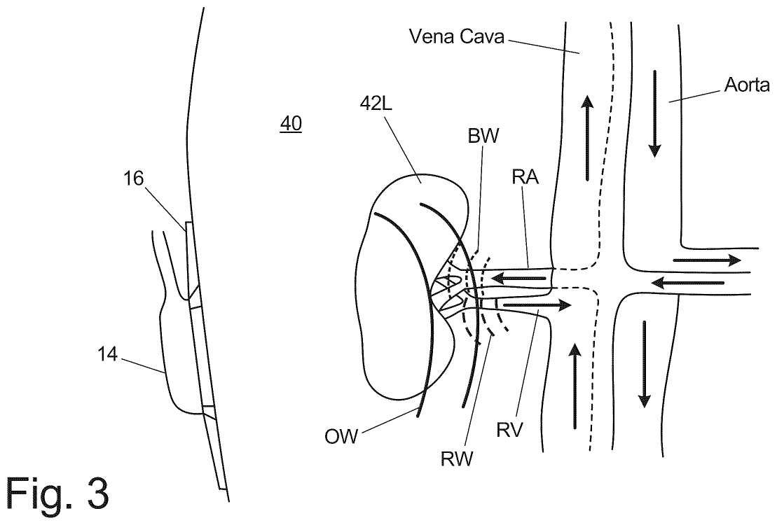

FIG. 3 is another schematic diagram of ultrasound transducer probe 14 attached to abdomen 40 of patient 10 by adhesive patch 16 over kidney 42L. The Doppler flow signal DF of kidney 42L can be measured from either the renal artery RA as blood enters kidney 42L from the aorta of patient 10 via the renal artery or from the renal vein RV as blood exits kidney 42L to the vena cava of patient 10 via the renal vein RV. Ultrasound transducer probe 14 generates originating signals OW that move into abdomen 40 of patient 10. Due to Doppler physics, a Doppler signal BW of the blood flow in the renal artery RA is “blue shifted” as the blood flow in the renal artery RA is moving toward the ultrasound transducer probe 14. A Doppler signal RW of the blood flow in the renal vein RV is “red shifted” as the blood flow in the renal vein RV is moving away from the ultrasound transducer. Since the Doppler signal BW is blue shifted and the Doppler signal RW is red shifted, renal blood flow monitor 12 can easily distinguish renal artery blood flow from renal vein blood flow. In human subjects the renal artery RA and renal vein RV are close and aligned parallel such that beamformer 48 can position the beam(s) to capture both arterial and venous flow of kidney 42L simultaneously.

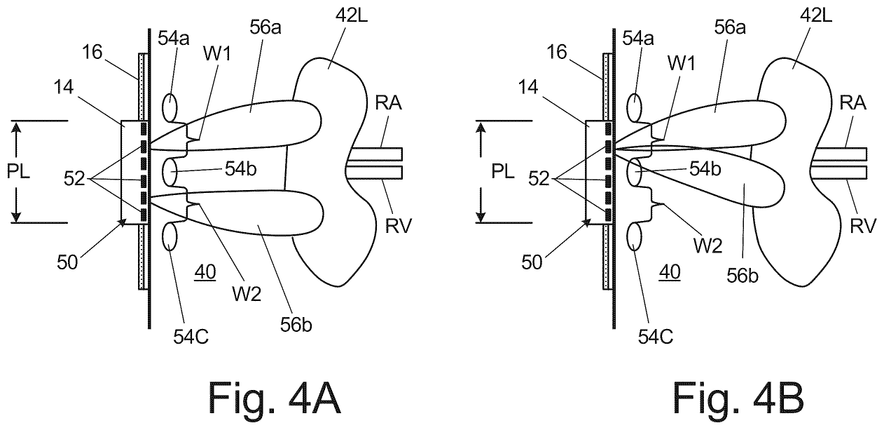

FIGS. 4A and 4B will be discussed concurrently. FIG. 4A is another schematic diagram of ultrasound transducer probe 14 attached to abdomen 40 of patient 10 by adhesive patch 16 over kidney 42L. FIG. 4B is also a schematic diagram of ultrasound transducer probe 14 attached to abdomen 40 of patient 10 by adhesive patch 16 over kidney 42L. In the example of FIGS. 4A and 4B, ultrasound transducer probe 14 is attached by adhesive patch 16 to a surface of abdomen 40 over kidney 42L and over at least some of ribs 54a, 54b, and 54c of patient 10.

Ultrasound transducer probe 14 can include a probe length PL, probe width PW (shown in FIG. 2), or diameter that is large enough that array 50 of transducer elements 52 of ultrasound transducer probe 14 can cover one or more acoustic windows in patient 10. An acoustic window of patient 10 is defined as an area of patient 10 where transmission of ultrasonic waves is not substantially attenuated in comparison to immediate surroundings. For example, array 50 of transducer elements 52 of ultrasound transducer probe 14 can be sized in length or width to extend over at least two intercostal spaces of patient 10. For example, in FIG. 4A, array 50 of transducer elements 52 of ultrasound transducer probe 14 is positioned over first acoustic window W1 (formed by the intercostal space between rib 54a and rib 54b) and over second acoustic window W2 (formed by the intercostal space between rib 54b and rib 54c. In the example of FIG. 4A, beamformer 48 (shown in FIG. 2) can selectively activate transducer elements 52 in array 50 to steer signal beams 56a and 56b into abdomen 40 through the first acoustic window W1 and/or second acoustic window W2 to avoid ribs 54a, 54b, and 54c. In the example of FIG. 4B, ultrasound transducer probe 14 is positioned slightly higher on abdomen 40 of patient 10 in comparison to the example of FIG. 4A. However, the probe length PL or probe width PW of ultrasound transducer probe 14 is long enough that ultrasound transducer probe 14 still has access to first acoustic window W1 and can still scan and steer signal beams 56a and 56b into abdomen 40 through the first acoustic window W1. Regardless of where ultrasound transducer probe 14 is placed over ribs 54a, 54b, and 54c, ribs 54a, 54b, and 54c will not block the direct view of kidney 42L from array 50 of ultrasound transducer probe 14.

Beamformer 48 controls transducer elements 52 in array 50 to beam scan abdomen 40 to find and sense the Doppler flow signal DF when ultrasound transducer probe 14 is first placed on patient 10. Beamformer 48 also controls transducer elements 52 in array 50 to track scan abdomen 40 to track the Doppler flow signal DF of the renal blood flow over time. Beamformer 48 beam scans and/or track scans the Doppler flow signal DF of the renal blood flow of kidney 42L of patient 10 by sequentially emitting signal beams 56a and 56b from array 50 of transducer elements 52 and focusing each of beams 56a and 56b in different locations. Signal beams 56a and 56b track the Doppler flow signal DF relative to array 50 of transducer elements 52. If kidney 42L, renal artery RA, and/or renal vein RV shifts within abdomen 40, the Doppler flow signal DF of the renal blood flow can be altered and decrease in signal strength. If that should happen, beamformer 48 can emit signal beam 56a and signal beam 56b (and possibly more signal beams) to scan and sweep about abdomen 40. In one example, beamformer 48 uses signal beams 56a and 56b to track a center of the renal blood flow where the Doppler flow signal DF is strongest and adjusts signal beams 56a and 56b to follow the center of the renal blood flow when the center moves and changes position.

In order for ultrasound transducer probe 14 to measure the Doppler flow signal DF of the renal blood flow of kidney 42L, ultrasound transducer probe 14 can have a low center frequency between 0.5 MHz and 4.0 MHz. With a center frequency between 0.5 MHz and 4.0 MHz, ultrasound transducer probe 14 can penetrate more than 15 cm into patient 10, which is a sufficient depth to measure the renal blood flow. This depth also allows ultrasound transducer probe 14 the ability to measure hepatic blood flow, celiac blood flow, portal blood flow, and mesenteric blood flow. Monitoring system 11 does not use ultrasound transducer probe 14 for high resolution imaging of kidney 42L. Thus, ultrasound transducer probe 14 can have a lower transducer element count than an ultrasound transducer probe used for ultrasound imaging. Lowering the transducer element count of array 50 of transducer elements 52 increases a signal-to-noise ratio SNR of ultrasound transducer probe 14.

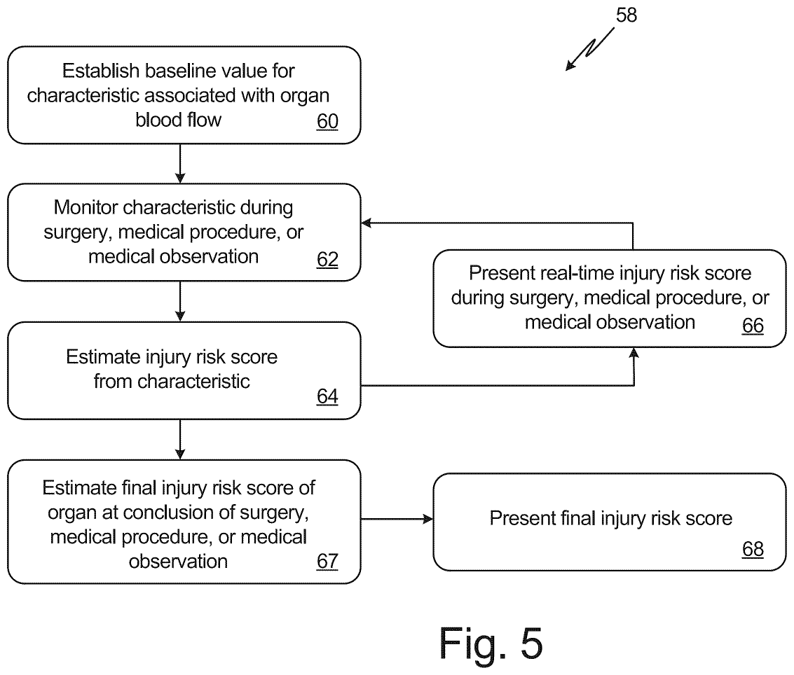

FIG. 5 is a block diagram of method 58 for operating monitoring system 11 shown in FIGS. 1-4B to continuously monitor a characteristic associated with a targeted organ blood flow of patient 10 during a surgery, medical procedure, or medical observation. Once ultrasound transducer probe 14 has been attached to patient 10 and is sensing a Doppler flow signal of the targeted organ blood flow, system processor 18 executes injury monitoring module 32 to perform first step 60 of method 58. In first step 60, system processor 18 executes injury monitoring module 32 to analyze the Doppler flow signal of the targeted organ blood flow for the characteristic and establish a baseline value for the characteristic. In second step 62 of method 58, system processor 18 executes injury monitoring module 32 to continuously monitor the Doppler flow signal of the targeted organ blood flow for the characteristic during the surgery, medical procedure, or medical observation of patient 10. As part of second step 62, system processor 18 can output a real time value for the characteristic to display 28. Display 28 can show a plot of the real time value for the characteristic over time.

In third step 64 of method 58, system processor 18 executes injury monitoring module 32 to estimate a real-time organ injury risk score of patient 10 from the characteristic. System processor 18 and injury monitoring module 32 can use the real time value of the characteristic, previously recorded values of the characteristic, and the baseline value of the characteristic to estimate the real-time organ injury risk score of patient 10. In fourth step 66 of method 58, system processor 18 outputs the real-time organ injury risk score of patient 10 to display 28. The real-time organ injury risk score can be shown on display 28 as a plot that shows how the real-time organ injury risk score of patient 10 changes over time, and/or the real-time organ injury risk score can be shown as a present value in injury score indicator 38. The real-time organ injury risk score is recorded by system processor 18 into system memory 20. When estimating a next iteration of the real-time organ injury risk score of patient 10, system processor 18 can use the recorded organ injury risk score(s) in system memory 20 as part of the estimation of the next iteration of the real-time organ injury risk score of patient 10. Thus, over time, the real-time organ injury risk score of patient 10 is based on both real-time information from the characteristic associated with the targeted organ blood flow plus cumulative past information of the characteristic.

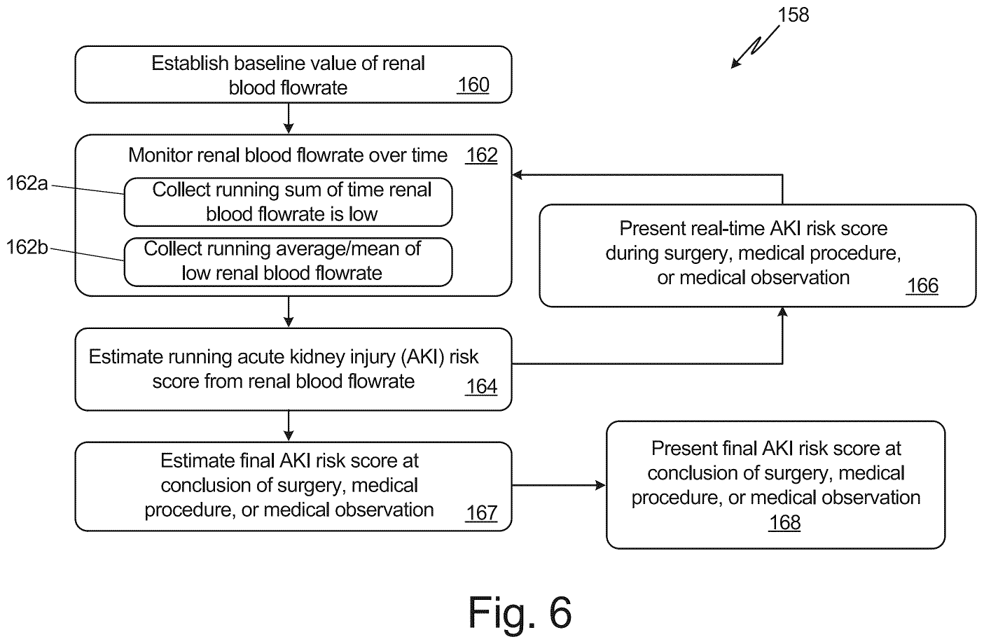

As the surgery, medical procedure, or medical observation of patient 10 progresses, system processor 18 and injury monitoring module 32 continues to repeat second step 62, third step 64, and fourth step 66 of method 58 to continuously update and display the real-time organ injury risk score of patient 10. At the end of the surgery, medical procedure, or medical observation of patient 10, system processor 18 can execute injury monitoring module 32 to perform fifth step 67 to estimate a final organ injury risk score of the targeted organ of patient 10. System processor 18 can determine the final organ injury risk score of patient 10 based on the values of the real-time organ injury risk score that were tracked and recorded to system memory 20 throughout the surgery, medical procedure, or medical observation of patient 10. After estimating the final organ injury risk score of the targeted organ, system processor 18 performs sixth step 68 of method 58 by outputting the final organ injury risk score to display 28. Based on the value of the final organ injury risk score, medical personnel can estimate if the targeted organ of patient 10 was injured during the surgery, medical procedure, or medical observation and can recommend that patient 10 seek treatment of the targeted organ. As discussed below with reference to FIGS. 6-8, the characteristic associated with the targeted organ blood flow of patient 10 can include, but is not limited to, a blood flow rate, a blood flow index, and an autoregulation profile of the targeted organ blood flow.

FIG. 6 is a block diagram of method 158 for operating monitoring system 11 shown in FIGS. 1-3 to continuously monitor a characteristic associated with a targeted organ blood flow of patient 10 during a surgery, medical procedure, or medical observation. Method 158 shown in FIG. 6 is an example of a specific application of method 58 shown in FIG. 5. In method 158, the targeted organ blood flow is a renal blood flow of kidney 42L. Renal blood flow rate is the characteristic associated with the renal blood flow that monitoring system 11 is continuously monitoring during a surgery, medical procedure, or medical observation of patient 10. Once ultrasound transducer probe 14 has been attached to patient 10 and is sensing the Doppler flow signal DF of the renal blood flow of kidney 42L, system processor 18 executes injury monitoring module 32 to perform first step 160 of method 158. In first step 160, system processor 18 executes injury monitoring module 32 to analyze the Doppler flow signal DF of the renal blood flow to determine the renal blood flow rate and establish a baseline value for the renal blood flow rate. System processor 18 can also execute injury monitoring module 32 to establish a threshold relative to the baseline value that defines when the renal blood flow rate has an abnormal value. An abnormal value of the renal blood flow rate is when the renal blood flow rate is below the baseline value. A low blood flow rate of the renal blood flow can be indicative of injury to kidney 42L. For example, the threshold can be 80% of the baseline value of the renal blood flow rate. Thus, when a real time value of the renal blood flow rate is less than or equal to 80% of the baseline value, the real time value of the renal blood flow rate is classified by system processor 18 and injury monitoring module 32 as being abnormal and low, or having a low value.

In second step 162 of method 158, system processor 18 executes injury monitoring module 32 to continuously monitor the Doppler flow signal DF of the renal blood flow for the renal blood flow rate during the surgery, medical procedure, or medical observation of patient 10. As part of second step 162, system processor 18 can output a real time value for the renal blood flow rate to display 28. Display 28 can show a plot of the real time value for the renal blood flow rate over time. In the example of FIG. 6, second step 162 of method 158 further includes first sub-step 162a and second sub-step 162b. In first sub-step 162a, system processor 18 executes injury monitoring module 32 to collect a running sum of time that the renal blood flow rate is low during the surgery, medical procedure, or medical observation of the patient. In second sub-step 162b, system processor 18 executes injury monitoring module 32 to collect a running average or a running mean or a time weighted average of the low values of the renal blood flow rate during the surgery, medical procedure, or medical observation of the patient.

In third step 164 of method 158, system processor 18 executes injury monitoring module 32 to estimate a real-time AKI risk score of patient 10 from the renal blood flow rate. System processor 18 and injury monitoring module 32 uses the real time value of the renal blood flow rate, the running sum of time that the renal blood flow rate was low, and the running average or the running mean or a time weighted average of the low values of the renal blood flow rate to estimate the real-time AKI risk score of patient 10. In fourth step 166 of method 158, system processor 18 outputs the real-time AKI risk score of patient 10 to display 28. The real-time AKI risk score can be shown on display 28 as a plot that shows how the real-time AKI risk score of patient 10 changes over time, and/or the real-time AKI risk score can be shown as a present value in injury score indicator 38. The real-time AKI risk score is recorded by system processor 18 into system memory 20. When estimating a next iteration of the real-time AKI risk score of patient 10, system processor 18 can use the recorded AKI risk score(s) in system memory 20 as part of the estimation of the next iteration of the real-time AKI risk score of patient 10. Thus, over time, the real-time AKI risk score of patient 10 is based on both real-time information from the characteristic associated with the targeted organ blood flow plus cumulative past information of the characteristic.

As the surgery, medical procedure, or medical observation of patient 10 progresses, system processor 18 and injury monitoring module 32 continues to repeat second step 162, third step 164, and fourth step 166 of method 158 to continuously update and display the real-time AKI risk score of patient 10. At the end of the surgery, medical procedure, or medical observation of patient 10, system processor 18 can execute injury monitoring module 32 to perform fifth step 167 to estimate a final AKI risk score of kidney 42L of patient 10. System processor 18 can determine the final AKI risk score of patient 10 based on the values of the real-time AKI risk score that were tracked and recorded to system memory 20 throughout the surgery, medical procedure, or medical observation of patient 10. After estimating the final AKI risk score of kidney 42L, system processor 18 performs sixth step 168 of method 158 by outputting the final AKI risk score to display 28. Based on the value of the final AKI risk score, medical personnel can estimate if kidney 42L of patient 10 was injured during the surgery, medical procedure, or medical observation and can recommend that patient 10 seek treatment of kidney 42L.

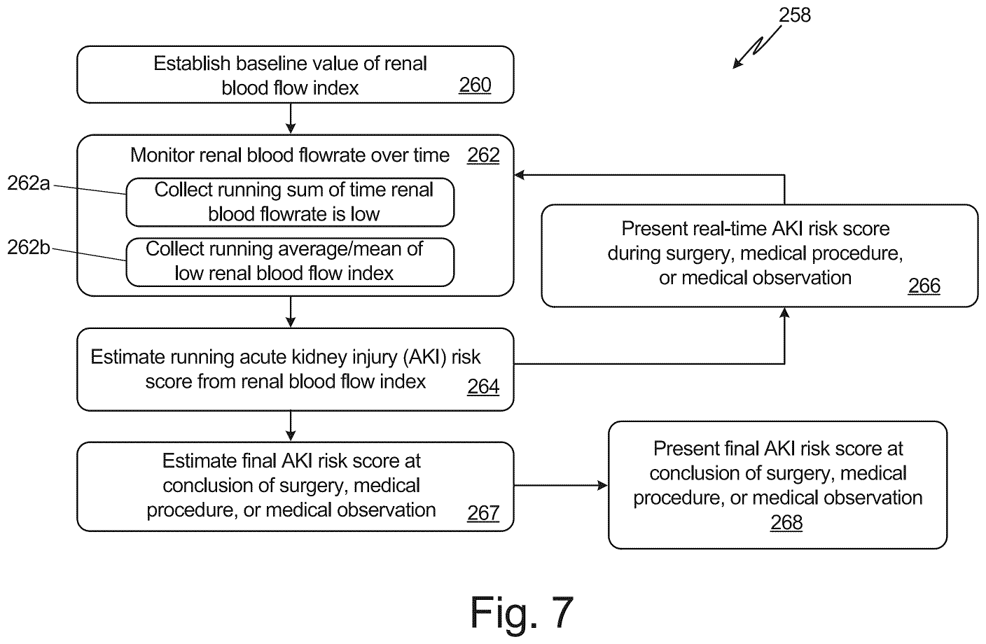

FIG. 7 is a block diagram of method 258 for operating the example of monitoring system 11 shown in FIGS. 1-3 to continuously monitor a characteristic associated with a targeted organ blood flow of patient 10 during a surgery, medical procedure, or medical observation. Method 258 shown in FIG. 7 is an example of method 58 shown in FIG. 5. In method 258, the targeted organ blood flow is the renal blood flow of kidney 42L. A normalized renal blood flow index is the characteristic in method 258 associated with the renal blood flow that monitoring system 11 is continuously monitoring during a surgery, medical procedure, or medical observation of patient 10. Injury monitoring module 32 includes software code that first estimates, when executed by system processor 18, a real-time renal blood flow index from the Doppler flow signal DF of the renal blood flow of kidney 42L and continuously outputs the real-time renal blood flow index to display 28. The real-time renal blood flow index can be estimated without normalization from various Doppler flow characteristics such as the intensity-weighted total or mean flow velocity over time, or the peak flow velocity.

Once ultrasound transducer probe 14 has been attached to patient 10 and is sensing the Doppler flow signal DF of the renal blood flow of kidney 42L, system processor 18 executes injury monitoring module 32 to perform first step 260 of method 258. In first step 260, system processor 18 executes injury monitoring module 32 to analyze the Doppler flow signal DF of the renal blood flow to determine the renal blood flow index of the renal blood flow of kidney 42L of patient 10. System processor 18 and injury monitoring module 32 can use the Renal Resistive Index (RRI) to calculate a normalized real-time renal blood flow index from the renal artery flow of kidney 42L. System processor 18 and injury monitoring module 32 can use Equation 1 to determine RRI from the Doppler flow signal DF of the renal blood flow of kidney 42L:

$$RRI = \frac{( peak systolic velocity - end diastolic velocity )}{peak systolic velocity} . \qquad Equation 1$$

System processor 18 and injury monitoring module 32 can also use or alternatively use a Venous Impedance Index (VII) to calculate a normalized real-time renal blood flow index from the Doppler flow signal DF of the renal blood flow in the renal vein of kidney 42L. System processor 18 and injury monitoring module 32 can use Equation 2 to determine VII from the Doppler flow signal DF of the renal blood flow of kidney 42L:

$$VII = \frac{( maximum flow velocity - minimum flow velocity )}{maximum flow velocity} . \qquad Equation 2$$

In first step 260, system processor 18 also executes injury monitoring module 32 to establish a baseline value for the renal blood flow index. For RRI, a value between 0.50-0.70 is considered a normal and healthy value. System processor 18 and injury monitoring module 32 can determine the baseline value by monitoring the normalized real-time renal blood flow index while patient 10 is under normal healthy conditions, or by choosing a baseline value established by past clinical studies, such as selecting a baseline RRI of 0.50-0.70.

System processor 18 can also execute injury monitoring module 32 to establish a threshold relative to the baseline value of the renal blood flow index that defines when the renal blood flow index has an abnormal value. The renal blood flow indices RRI and VII have an abnormal value when the renal blood flow index is above the threshold. A high blood flow index of the renal blood flow can be indicative of injury to kidney 42L. For example, the threshold can be 120% of the baseline value of the renal blood flow index. Thus, when a real time value of the renal blood flow index is greater than or equal to 120% of the baseline value of the renal blood flow index, the real time value of the renal blood flow index is classified by system processor 18 and injury monitoring module 32 as being high or having a high value. Other renal blood flow indices may have abnormal values below a threshold or outside of a normal range defined by a high or low value

In second step 262 of method 258, system processor 18 executes injury monitoring module 32 to continuously monitor the Doppler flow signal DF of the renal blood flow for the renal blood flow index during the surgery, medical procedure, or medical observation of patient 10. As part of second step 262, system processor 18 can output a real time value for the renal blood flow index to display 28. Display 28 can show a plot of the real time value for the renal blood flow index over time. In the example of FIG. 7, second step 262 of method 258 further includes first sub-step 262a and second sub-step 262b. In first sub-step 262a, system processor 18 executes injury monitoring module 32 to collect a running sum of time that the renal blood flow index is high during the surgery, medical procedure, or medical observation of the patient. In second sub-step 262b, system processor 18 executes injury monitoring module 32 to collect a running average or a running mean of the high values of the renal blood flow index during the surgery, medical procedure, or medical observation of the patient.

In third step 264 of method 258, system processor 18 executes injury monitoring module 32 to estimate a real-time AKI risk score of kidney 42L of patient 10 from the renal blood flow index of the renal blood flow of kidney 42L. System processor 18 and injury monitoring module 32 uses the real time value of the renal blood flow index, the running sum of time that the renal blood flow index was high, and the running average or the running mean of the high values of the renal blood flow index to estimate the real-time AKI risk score of patient 10. In fourth step 266 of method 258, system processor 18 outputs the real-time AKI risk score of patient 10 to display 28. The real-time AKI risk score can be shown on display 28 as a plot that shows how the real-time AKI risk score of patient 10 changes over time, and/or the real-time AKI risk score can be shown as a present value in injury score indicator 38. The real-time AKI risk score is recorded by system processor 18 into system memory 20. When estimating a next iteration of the real-time AKI risk score of patient 10, system processor 18 can use the recorded AKI risk score(s) in system memory 20 as part of the estimation of the next iteration of the real-time AKI risk score of patient 10. Thus, over time, the real-time AKI risk score of patient 10 is based on both real-time information from the characteristic associated with the targeted organ blood flow plus cumulative past information of the characteristic.

As the surgery, medical procedure, or medical observation of patient 10 progresses, system processor 18 and injury monitoring module 32 continues to repeat second step 262, third step 264, and fourth step 266 of method 258 to continuously update and display the real-time AKI risk score of patient 10. At the end of the surgery, medical procedure, or medical observation of patient 10, system processor 18 can execute injury monitoring module 32 to perform fifth step 267 to estimate a final AKI risk score of kidney 42L of patient 10. System processor 18 can determine the final AKI risk score of patient 10 based on the values of the real-time AKI risk score that were tracked and recorded to system memory 20 throughout the surgery, medical procedure, or medical observation of patient 10. After estimating the final AKI risk score of kidney 42L, system processor 18 performs sixth step 268 of method 258 by outputting the final AKI risk score to display 28. Based on the value of the final AKI risk score, medical personnel can estimate if kidney 42L of patient 10 was injured during the surgery, medical procedure, or medical observation and can recommend that patient 10 seek treatment of kidney 42L.

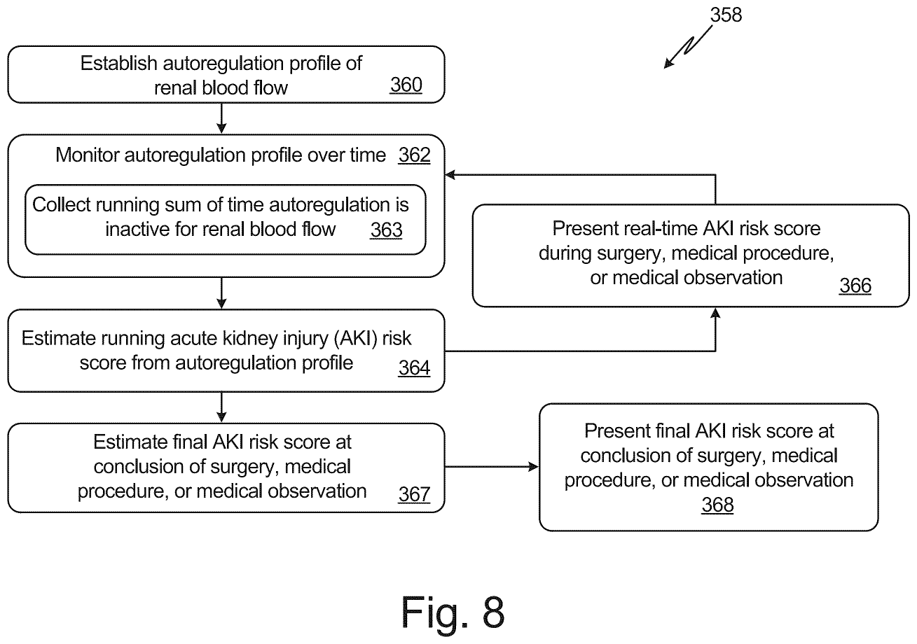

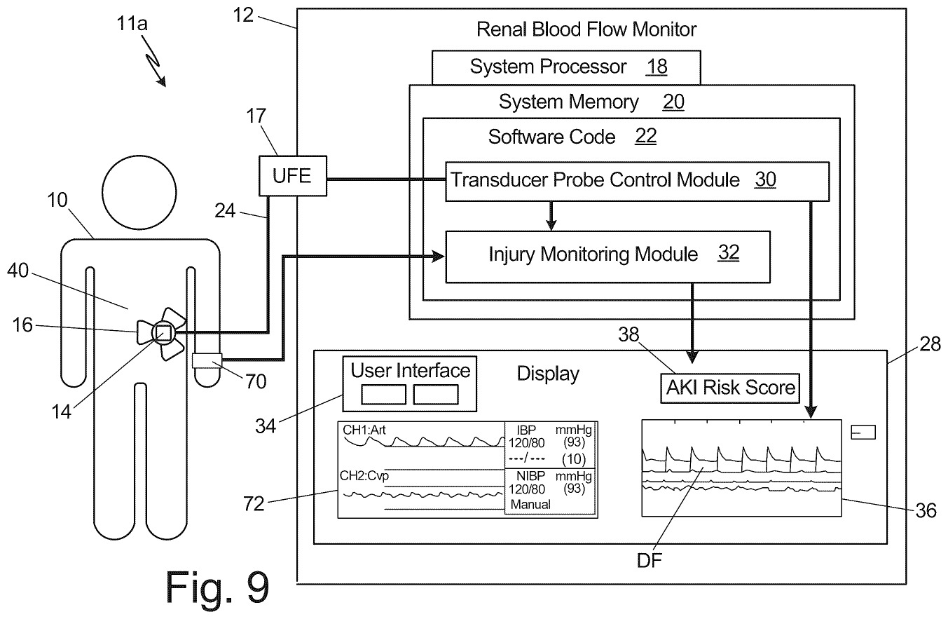

FIGS. 8 and 9 will be discussed concurrently. FIG. 8 is a block diagram of method 358 for operating the example of monitoring system 11 shown in FIGS. 1-3 to continuously monitor a characteristic associated with a targeted organ blood flow of patient 10 during a surgery, medical procedure, or medical observation. Method 358 shown in FIG. 8 is an example of method 58 shown in FIG. 5. In method 358, the targeted organ blood flow is the renal blood flow of kidney 42L. Autoregulation or an autoregulation profile is the characteristic associated with the renal blood flow that monitoring system 11 is continuously monitoring during a surgery, medical procedure, or medical observation of patient 10. Autoregulation of the renal blood flow of kidney 42L is defined as the ability of the renal arteries and the renal veins to dilate and constrict in response to dynamic perfusion pressure changes to maintain the renal blood flow sufficient to the organ's needs. Depending upon the state of the organ and subject's physiology, this often means a relatively constant blood flow despite changes in perfusion pressure, in other situations it can result in changes in the organ blood flow to meet metabolic or other requirements of the organ. In either case, the changes in blood flow of the organ are largely uncorrelated with changes in blood pressure.

FIG. 9 is a schematic diagram illustrating an example monitoring system 11a for performing method 358. Monitoring system 11a of FIG. 9 is similar to monitoring system 11 of FIG. 1 with the addition of pressure sensor 70. Monitoring system 11a includes blood flow monitor 12 with ultrasound transducer probe 14 attached to abdomen 40 of patient 10 by adhesive patch 16. Similar to monitoring system 11 of FIG. 1, monitoring system 11a uses ultrasound transducer probe 14 to monitor renal blood flow into kidney 42L of patient 10. Pressure sensor 70 of monitoring system 11a is attached to patient 10 for sensing hemodynamic data representative of an arterial pressure 72 of patient 10.

Monitoring system 11a uses the arterial pressure 72 of patient 10 and a renal flood flow rate of kidney 42L estimated from the Doppler flow signal DF to determine the autoregulation profile of kidney 42L of patient 10. System processor 18 monitors changes in the time domain and/or changes in the frequency domain for both the renal blood flow rate and the arterial pressure 72 of patient 10. System processor 18 evaluates relative to one another the changes in the renal blood flow rate and the changes in the arterial pressure 72 to determine the autoregulation profile of kidney 42L of patient 10. If system processor 18 determines a non-correlation between changes in the renal blood flow rate and changes in the arterial pressure 72 of patient 10, then system processor 18 determines that the autoregulation profile of kidney 42L is active and functioning properly. If system processor 18 determines a correlation between changes in the renal blood flow rate and changes in the arterial pressure 72 of patient 10, then system processor 18 determines that the autoregulation profile of kidney 42L is inactive and not functioning properly. The Pearson Correlation Coefficient is an example of a time domain correlation that system processor 18 can use over a rolling time window to monitor the renal blood flow rate and the arterial pressure 72 of patient 10 for autoregulation. The Coherence Function, sometimes referred to as the Magnitude-Squared Coherence Function, is an example of a frequency domain correlation that system processor 18 can use to monitor the renal blood flow rate and the arterial pressure 72 of patient 10 for autoregulation.

Pressure sensor 70 is operatively connected to blood flow monitor 12 (e.g., electrically and/or communicatively connected via wired or wireless connection, or both) to provide the sensed hemodynamic data to blood flow monitor 12 as analog sensor data (or as a digitized representation of the analog sensor data). As shown in FIG. 9, system processor 18 of blood flow monitor 12 can output a waveform plot of the arterial pressure 72 to display 28. In some examples, pressure sensor 70 can be attached non-invasively at an extremity of patient 10, such as a wrist, an arm, a finger, an ankle, a toe, or other extremity of patient 10. As such, pressure sensor 70 can take the form of a small, lightweight, and comfortable hemodynamic sensor suitable for extended wear by patient 10 to provide substantially continuous beat-to-beat monitoring of the arterial pressure 72 of patient 10 over an extended period of time, such as minutes or possibly hours. In certain examples, pressure sensor 70 can be configured to sense an arterial pressure 72 of patient 10 in a minimally invasive manner. For instance, pressure sensor 70 can be attached to patient 10 via a radial arterial catheter inserted into an arm of patient 10. In other examples, pressure sensor 70 can be attached to patient 10 via a femoral arterial catheter inserted into a leg of patient 10. Such minimally invasive techniques can similarly enable pressure sensor 70 to provide substantially continuous beat-to-beat monitoring of the arterial pressure 72 of patient 10 over an extended period of time, such as minutes or hours.

Once ultrasound transducer probe 14 has been attached to patient 10 and is sensing the Doppler flow signal DF of the renal blood flow of kidney 42L and pressure sensor 70 has been attached to patient 10 and is sensing hemodynamic data representative of an arterial pressure 72 of patient 10, system processor 18 executes injury monitoring module 32 to perform first step 360 of method 358. In first step 360, system processor 18 executes injury monitoring module 32 to analyze the Doppler flow signal DF of the renal blood flow and the arterial pressure 72 of patient 10 to determine the autoregulation profile of the renal blood flow of kidney 42L and establish how the autoregulation profile appears when the autoregulation of the renal blood flow is active and how the autoregulation profile appears when the autoregulation of the renal blood flow is inactive. Inactive autoregulation of the renal blood flow to kidney 42L over time can be indicative of injury to kidney 42L.

In second step 362 of method 358, system processor 18 executes injury monitoring module 32 to continuously monitor the Doppler flow signal DF of the renal blood flow for the autoregulation profile of the renal blood flow to kidney 42L during the surgery, medical procedure, or medical observation of patient 10. As part of second step 362, system processor 18 can output the autoregulation profile of the renal blood flow to display 28. Display 28 can show a plot of the autoregulation profile for the renal blood flow of kidney 42L over time. In the example of FIG. 8, second step 362 of method 358 further includes sub-step 363. In sub-step 363, system processor 18 executes injury monitoring module 32 to collect a running sum of time that the autoregulation profile indicates that the autoregulation of the renal blood flow of kidney 42L is inactive during the surgery, the medical procedure, or the medical observation of patient 10.

In third step 364 of method 358, system processor 18 executes injury monitoring module 32 to estimate a real-time AKI risk score of patient 10 from the autoregulation profile of the renal blood flow. System processor 18 and injury monitoring module 32 use the running sum of the time that the autoregulation of the renal blood flow was inactive to estimate the real-time AKI risk score of patient 10. In fourth step 366 of method 358, system processor 18 outputs the real-time AKI risk score of patient 10 to display 28. The real-time AKI risk score can be shown on display 28 as a plot that shows how the real-time AKI risk score of patient 10 changes over time, and/or the real-time AKI risk score can be shown as a present value in injury score indicator 38. The real-time AKI risk score is recorded by system processor 18 into system memory 20. When estimating a next iteration of the real-time AKI risk score of patient 10, system processor 18 can use the recorded AKI risk score(s) in system memory 20 as part of the estimation of the next iteration of the real-time AKI risk score of patient 10. Thus, over time, the real-time AKI risk score of patient 10 is based on both real-time information from the characteristic associated with the targeted organ blood flow plus cumulative past information of the characteristic.

As the surgery, medical procedure, or medical observation of patient 10 progresses, system processor 18 and injury monitoring module 32 continues to repeat second step 362, third step 364, and fourth step 366 of method 358 to continuously update and display the real-time AKI risk score of patient 10. Whenever monitoring system 11 indicates that the autoregulation of the renal blood flow of kidney 42L is inactive, monitoring system 11 can activate an alert or alarm to make medical personnel aware so that the medical personnel can take action to compensate for the inactive autoregulation or take action to restore autoregulation of the renal blood flow. At the end of the surgery, medical procedure, or medical observation of patient 10, system processor 18 can execute injury monitoring module 32 to perform fifth step 367 to estimate a final AKI risk score of kidney 42L of patient 10. System processor 18 can determine the final AKI risk score of patient 10 based on the values of the real-time AKI risk score that were tracked and recorded to system memory 20 throughout the surgery, medical procedure, or medical observation of patient 10. After estimating the final AKI risk score of kidney 42L, system processor 18 performs sixth step 368 of method 358 by outputting the final AKI risk score to display 28. Based on the value of the final AKI risk score, medical personnel can estimate if kidney 42L of patient 10 was injured during the surgery, medical procedure, or medical observation and can recommend that patient 10 seek treatment of kidney 42L.

In some examples, monitoring system 11 can monitor more than one characteristic of the renal blood flow to determine the real-time AKI risk score and the final AKI risk score of kidney 42L of patient 10. For example, monitoring system 11 can monitor two or more of the renal blood flow rate, the renal blood flow index, and the autoregulation profile of the renal blood flow of kidney 42L to determine the real-time AKI risk score and the final AKI risk score of kidney 42L. Monitoring system 11 is also not limited to using the renal blood flow rate, the renal blood flow index, and the autoregulation profile of the renal blood flow of kidney 42L as the sole characteristics of the renal blood flow for determining the real-time AKI risk score and the final AKI risk score of kidney 42L. Additional characteristics associated with the renal blood flow and determined from the Doppler flow signal DF of the renal blood flow can be used by monitoring system 11 to determine the real-time AKI risk score and the final AKI risk score of kidney 42L.

### Discussion of Possible Embodiments

The following are non-exclusive descriptions of possible embodiments of the present invention.

A method is disclosed for monitoring a patient with a blood flow monitor in communication with an ultrasound transducer probe during a surgery, a medical procedure, or a medical observation. The method includes obtaining a Doppler flow signal of a renal blood flow of the patient with the ultrasound transducer probe attached in a stationary position to an abdomen to the patient. A processor of the blood flow monitor determines a characteristic associated with the renal blood flow of the patient from the Doppler flow signal. The processor determines a baseline value of the characteristic and continuously monitors over time the Doppler flow signal and the characteristic during the surgery, medical procedure, or medical observation of the patient.

The method of the preceding paragraph can optionally include, additionally and/or alternatively, any one or more of the following features, configurations and/or additional components in the paragraphs below.

In an embodiment of the foregoing method, the method further comprises: positioning the ultrasound transducer probe on the abdomen of the patient and attaching the ultrasound transducer probe to the abdomen of the patient with an adhesive patch to maintain contact between the ultrasound transducer probe and the patient without an ultrasound operator; and scanning the abdomen of the patient with the ultrasound transducer probe to find the Doppler flow signal of the renal blood flow of the patient.

In an embodiment of the foregoing method, the method further comprises outputting in real time to a display in communication with the processor a representation of the Doppler flow signal over time and a representation of the characteristic over time.

In an embodiment of the foregoing method, continuously monitoring over time the Doppler flow signal and the characteristic during the surgery, medical procedure, or medical observation of the patient comprises: collecting, by the blood flow monitor, a running sum of time that the characteristic is below the baseline value of the characteristic during the surgery, medical procedure, or medical observation of the patient.

In an embodiment of the foregoing method, continuously monitoring over time the Doppler flow signal and the characteristic during the surgery, medical procedure, or medical observation of the patient comprises: collecting, by the processor of the blood flow monitor, a running average or running mean of abnormal values of the characteristic during the surgery, medical procedure, or medical observation of the patient.

In an embodiment of the foregoing method, the characteristic associated with the renal blood flow of the patient from the Doppler flow signal comprises a flow rate of the renal blood flow of the patient.

In an embodiment of the foregoing method, the characteristic associated with the renal blood flow of the patient from the Doppler flow signal comprises a renal blood flow index of the renal blood flow of the patient.

In an embodiment of the foregoing method, the renal blood flow index of the renal blood flow of the patient comprises a Venous Impedance Index (VII) and/or a Renal Resistive Index (RRI).

In an embodiment of the foregoing method, the characteristic associated with the renal blood flow of the patient from the Doppler flow signal comprises an autoregulation profile of the renal blood flow of the patient.

In an embodiment of the foregoing method, the method further comprises: obtaining an arterial pressure of the patient with a pressure sensor attached to the patient; estimating by the processor of the blood flow monitor a flow rate of the renal blood flow of the patient from the Doppler flow signal of the renal blood flow; monitoring by the processor changes in the flow rate of the renal blood flow over time; monitoring by the processor changes in the arterial pressure over time; evaluating a correlation or non-correlation by the processor between the changes in the arterial pressure and the changes in the flow rate of the renal blood flow; and determining by the processor the autoregulation profile of the renal blood flow of the patient based on the correlation or non-correlation between the changes in the arterial pressure and the changes in the flow rate of the renal blood flow.

In an embodiment of the foregoing method, the pressure sensor is attached to the patient by a radial arterial catheter.

In an embodiment of the foregoing method, the pressure sensor is attached to the patient by a femoral arterial catheter.

In an embodiment of the foregoing method, the pressure sensor is attached non-invasively to an extremity of the patient.

In an embodiment of the foregoing method, the method further comprises: estimating, by the processor of the blood flow monitor, a real-time acute kidney injury risk score of the patient from the characteristic and the baseline value of the characteristic; and outputting in real time to the display a representation of the real-time acute kidney injury risk score of the patient over time.

In an embodiment of the foregoing method, tracking over time, by the processor, the real-time acute kidney injury risk score of the patient to determine a final acute kidney injury risk score of the patient.

In an embodiment of the foregoing method, the method further comprises: executing beamformer software code by the processor to track-scan the Doppler flow signal of the renal blood flow of the patient with a two-dimensional phased array of transducer elements of the ultrasound transducer probe to continuously sense the Doppler flow signal of the renal blood flow of the patient during the surgery, medical procedure, or medical observation without an ultrasound operator.

In an embodiment of the foregoing method, the method further comprises: executing the beamformer software code by the processor to emit a set of sequential beams from the array of transducer elements to track a center of the renal blood flow relative to the array of transducer elements; focusing, by the processor and the beamformer software code, each beam from the set of sequential beams in different locations; and adjusting, by the processor and the beamformer software code, the position of the set of sequential beams onto the center of the renal blood flow to maintain the Doppler flow signal of the renal blood flow of the patient.

A system includes an ultrasound transducer probe with a two-dimensional array of transducer elements configured to measure a Doppler flow signal of a renal blood flow of a patient. An adhesive patch is connected to the ultrasound transducer probe and is configured to attach the ultrasound transducer probe to the patient and maintain contact between the patient and the ultrasound transducer probe without an operator. A renal blood flow monitor is in communication with the ultrasound transducer probe. The renal blood flow monitor includes a system memory that stores monitoring software code. The renal blood flow monitor also includes a processor configured to execute the monitoring software code to determine a characteristic associated with the renal blood flow of the patient and monitor over time the characteristic associated with the renal blood flow of the patient.

The system of the preceding paragraph can optionally include, additionally and/or alternatively, any one or more of the following features, configurations and/or additional components in the paragraphs below.

In an embodiment of the foregoing system, the system further comprises: a display in communication with the processor to receive and show a continuous reading of the Doppler flow signal from the ultrasound transducer probe and a representation of the characteristic over time.

In an embodiment of the foregoing system, the processor is configured to execute the monitoring software code to: determine a baseline value of the characteristic associated with the renal blood flow of the patient.

In an embodiment of the foregoing system, the characteristic associated with the renal blood flow of the patient from the Doppler flow signal comprises a flow rate of the renal blood flow of the patient.

In an embodiment of the foregoing system, the characteristic associated with the renal blood flow of the patient from the Doppler flow signal comprises a renal blood flow index of the renal blood flow of the patient.

In an embodiment of the foregoing system, the renal blood flow index of the renal blood flow of the patient comprises a Venous Impedance Index (VII) and/or a Renal Resistive Index (RRI).

In an embodiment of the foregoing system, the characteristic associated with the renal blood flow of the patient from the Doppler flow signal comprises an autoregulation profile of the renal blood flow of the patient.

In an embodiment of the foregoing system, the system further comprises: a hemodynamic pressure sensor configured to measure an arterial pressure of the patient, wherein the processor is configured to monitor the arterial pressure of the patient measured by the hemodynamic pressure sensor, and wherein the processor is configured to determine the autoregulation profile of the renal blood flow of the patient based on changes over time in a flow rate of the renal blood flow determined from the Doppler flow signal over time and based on changes over time in the arterial pressure of the patient measured by the hemodynamic pressure sensor.

In an embodiment of the foregoing system, the hemodynamic pressure sensor is connected to a radial arterial catheter.

In an embodiment of the foregoing system, the hemodynamic pressure sensor is connected to a femoral arterial catheter.

In an embodiment of the foregoing system, the hemodynamic pressure sensor is a non-invasive hemodynamic pressure sensor.

In an embodiment of the foregoing system, the processor is configured to execute the monitoring software code to: estimate a real-time acute kidney injury risk score of the patient from the characteristic; and output in real time to the display a representation of the real-time acute kidney injury risk score of the patient over time.

In an embodiment of the foregoing system, the processor is configured to execute the monitoring software code to: track over time the real-time acute kidney injury risk score of the patient to determine a final acute kidney injury risk score of the patient.

In an embodiment of the foregoing system, the two-dimensional array of transducer elements of the ultrasound transducer probe comprises a phased array of transducer elements.

In an embodiment of the foregoing system, the system memory stores probe control software code with beamformer software code, and wherein the processor is configured to execute the beamformer software code to: to track-scan the Doppler flow signal of the renal blood flow of the patient by emitting multiple ultrasound beams from the phased array of transducer elements to track the Doppler flow signal of the renal blood flow of the patient relative to the phased array of transducer elements.

A method is disclosed for monitoring a patient during a surgery, a medical procedure, or a medical observation with a blood flow monitor in communication with an ultrasound transducer probe. The method includes obtaining a Doppler flow signal of a targeted organ blood flow of the patient with the ultrasound transducer probe attached in a stationary position to an abdomen of the patient. A processor of the blood flow monitor determines at least one characteristic associated with the targeted organ blood flow of the patient from the Doppler flow signal. The processor determines a baseline value of the at least one characteristic and continuously monitors over time the Doppler flow signal and the at least one characteristic during the surgery, medical procedure, or medical observation of the patient.

The method of the preceding paragraph can optionally include, additionally and/or alternatively, any one or more of the following features, configurations and/or additional components in the paragraphs below.

In an embodiment of the foregoing method, the method further comprises: positioning the ultrasound transducer probe on the abdomen of the patient and attaching the ultrasound transducer probe to the abdomen of the patient with an adhesive patch to maintain contact between the ultrasound transducer probe and the patient without an ultrasound operator; and scanning the abdomen of the patient with the ultrasound transducer probe to find the Doppler flow signal of the targeted organ blood flow of the patient.

In an embodiment of the foregoing method, the method further comprises outputting in real time to a display in communication with the processor a representation of the Doppler flow signal over time and a representation of the at least one characteristic over time.

In an embodiment of the foregoing method, continuously monitoring over time the Doppler flow signal and the at least one characteristic during the surgery, medical procedure, or medical observation of the patient comprises: collecting, by the blood flow monitor, a running sum of time that the at least one characteristic is below the baseline value of the at least one characteristic during the surgery, medical procedure, or medical observation of the patient.

In an embodiment of the foregoing method, continuously monitoring over time the Doppler flow signal and the at least one characteristic during the surgery, medical procedure, or medical observation of the patient comprises: collecting, by the processor of the blood flow monitor, a running average, or a running mean, of abnormal values of the at least one characteristic during the surgery, medical procedure, or medical observation of the patient.

In an embodiment of the foregoing method, the at least one characteristic associated with the targeted organ blood flow of the patient from the Doppler flow signal comprises a flow rate of the targeted organ blood flow of the patient.

In an embodiment of the foregoing method, the at least one characteristic associated with the targeted organ blood flow of the patient from the Doppler flow signal comprises a blood flow index of the targeted organ blood flow of the patient.

In an embodiment of the foregoing method, the targeted organ blood flow is a renal blood flow of the patient, and the blood flow index comprises a Venous Impedance Index (VII) and/or a Renal Resistive Index (RRI).

In an embodiment of the foregoing method, the at least one characteristic associated with the targeted organ blood flow of the patient from the Doppler flow signal comprises an autoregulation profile of the targeted organ blood flow of the patient.

In an embodiment of the foregoing method, renal blood flow of the patient is the targeted organ blood flow, and the method further comprises: obtaining an arterial pressure of the patient with a pressure sensor attached to the patient; estimating by the processor of the blood flow monitor a flow rate of the renal blood flow of the patient from the Doppler flow signal of the renal blood flow; and monitoring by the processor changes in the flow rate of the renal blood flow over time; monitoring by the processor changes in the arterial pressure over time; evaluating a correlation or non-correlation by the processor between the changes in the arterial pressure and the changes in the flow rate of the renal blood flow; and determining by the processor the autoregulation profile of the renal blood flow of the patient based on the correlation or non-correlation between the changes in the arterial pressure and the changes in the flow rate of the renal blood flow.

In an embodiment of the foregoing method, the pressure sensor is attached to the patient by a radial arterial catheter.

In an embodiment of the foregoing method, the pressure sensor is attached to the patient by a femoral arterial catheter.

In an embodiment of the foregoing method, the pressure sensor is attached non-invasively to an extremity of the patient.

In an embodiment of the foregoing method, the method further comprises estimating, by the processor of the blood flow monitor, a real-time organ injury risk score of the patient from the at least one characteristic and the baseline value of the at least one characteristic; and outputting in real time to the display a representation of the real-time organ injury risk score of the patient over time.

In an embodiment of the foregoing method, the method further comprises tracking over time, by the processor, the real-time organ injury risk score of the patient to determine a final organ injury risk score of the patient.

In an embodiment of the foregoing method, the method further comprises executing beamformer software code by the processor to track-scan the Doppler flow signal of the targeted organ blood flow of the patient with a two-dimensional phased array of transducer elements of the ultrasound transducer probe to continuously sense the Doppler flow signal of the targeted organ blood flow of the patient during the surgery, medical procedure, or medical observation without an ultrasound operator.

In an embodiment of the foregoing method, the method further comprises: executing the beamformer software code by the processor to emit a set of sequential beams from the array of transducer elements into the abdomen of the patient to track a center of the targeted organ blood flow relative to the array of transducer elements; focusing, by the processor and the beamformer software code, each beam from the set of sequential beams in different locations; and adjusting, by the processor and the beamformer software code, the position of the set of sequential beams onto the center of the targeted organ blood flow to maintain the Doppler flow signal of the targeted organ blood flow of the patient.

A system includes an ultrasound transducer probe with a two-dimensional array of transducer elements configured to measure a Doppler flow signal of a targeted organ blood flow of a patient. An adhesive patch is connected to the ultrasound transducer probe and is configured to attach the ultrasound transducer probe to the patient and maintain contact between the patient and the ultrasound transducer probe without an operator. A blood flow monitor is in communication with the ultrasound transducer probe. The blood flow monitor includes a processor and system memory that stores monitoring software code. The processor is configured to execute the monitoring software code to determine at least one characteristic associated with the targeted organ blood flow of the patient. The processor is also configured to execute the monitoring software code to monitor over time the at least one characteristic associated with the targeted organ blood flow of the patient during a surgery, medical procedure, or medical observation of the patient.

The system of the preceding paragraph can optionally include, additionally and/or alternatively, any one or more of the following features, configurations and/or additional components in the paragraphs below.

In an embodiment of the foregoing system, the system further comprises: a display in communication with the processor to receive and show a continuous reading of the Doppler flow signal from the ultrasound transducer probe and a representation of the at least one characteristic over time.

In an embodiment of the foregoing system, the processor is configured to execute the monitoring software code to: determine a baseline value of the at least one characteristic associated with the targeted organ blood flow of the patient.

In an embodiment of the foregoing system, the at least one characteristic associated with the targeted organ blood flow of the patient from the Doppler flow signal comprises a flow rate of the targeted organ blood flow of the patient.

In an embodiment of the foregoing system, the at least one characteristic associated with the targeted organ blood flow of the patient from the Doppler flow signal comprises a blood flow index of the targeted organ blood flow of the patient.

In an embodiment of the foregoing system, the targeted organ blood flow is a renal blood flow of the patient, and wherein the blood flow index comprises a Venous Impedance Index (VII) and/or a Renal Resistive Index (RRI).

In an embodiment of the foregoing system, the at least one characteristic associated with the targeted organ blood flow of the patient from the Doppler flow signal comprises an autoregulation profile of the targeted organ blood flow of the patient.

In an embodiment of the foregoing system, the processor is configured to execute the monitoring software code to: collect a running sum of a duration of inactive autoregulation of the targeted organ blood flow of the patient during the surgery, the medical procedure, or the medical observation.

In an embodiment of the foregoing system, the system further comprises a hemodynamic pressure sensor configured to measure an arterial pressure of the patient, wherein the processor is configured to monitor the arterial pressure of the patient measured by the hemodynamic pressure sensor, and wherein the processor is configured to determine the autoregulation profile of the targeted organ blood flow of the patient based on changes in the targeted organ blood flow determined from the Doppler flow signal over time and based on changes in the arterial pressure of the patient over time measured by the hemodynamic pressure sensor.

In an embodiment of the foregoing system, the hemodynamic pressure sensor is connected to a radial arterial catheter.

In an embodiment of the foregoing system, the hemodynamic pressure sensor is connected to a femoral arterial catheter.

In an embodiment of the foregoing system, the hemodynamic pressure sensor is a non-invasive hemodynamic pressure sensor.

In an embodiment of the foregoing system, the monitoring software code comprises organ injury monitoring software code, and wherein the processor is configured to execute the organ injury monitoring software code to: estimate a real-time organ injury risk score of the patient from the at least one characteristic; and output in real time to the display a representation of the real-time organ injury risk score of the patient over time.

In an embodiment of the foregoing system, the processor is configured to execute the organ injury monitoring software code to: track over time the real-time organ injury risk score of the patient to determine a final organ injury risk score of the patient.

In an embodiment of the foregoing system, the two-dimensional array of transducer elements of the ultrasound transducer probe comprises a phased array of transducer elements.

In an embodiment of the foregoing system, the system memory stores probe control software code with beamformer software code, and wherein the processor is configured to execute the beamformer software code to: to track-scan the Doppler flow signal of the targeted organ blood flow of the patient by emitting multiple ultrasound beams from the phased array of transducer elements to track the Doppler flow signal of the targeted organ blood flow of the patient relative to the phased array of transducer elements.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method for monitoring a patient during a surgery, a medical procedure, or a medical observation with a blood flow monitor in communication with an ultrasound transducer probe, the method comprising:
obtaining a Doppler flow signal of a targeted organ blood flow of the patient with the ultrasound transducer probe attached in a stationary position to an abdomen of the patient;
determining by a processor of the blood flow monitor at least one characteristic associated with the targeted organ blood flow of the patient from the Doppler flow signal;
determining, by the processor, a baseline value of the at least one characteristic; and
continuously monitoring over time by the processor of the blood flow monitor the Doppler flow signal and the at least one characteristic during the surgery, medical procedure, or medical observation of the patient.

2. The method of claim 1, further comprising:
positioning the ultrasound transducer probe on the abdomen of the patient and attaching the ultrasound transducer probe to the abdomen of the patient with an adhesive patch to maintain contact between the ultrasound transducer probe and the patient without an ultrasound operator; and
scanning the abdomen of the patient with the ultrasound transducer probe to find the Doppler flow signal of the targeted organ blood flow of the patient.

3. The method of claim 2, further comprising:
outputting in real time to a display in communication with the processor a representation of the Doppler flow signal over time and a representation of the at least one characteristic over time.

4. The method of claim 1, wherein continuously monitoring over time the Doppler flow signal and the at least one characteristic during the surgery, medical procedure, or medical observation of the patient comprises:
collecting, by the blood flow monitor, a running sum of time that the at least one characteristic is below the baseline value of the at least one characteristic during the surgery, medical procedure, or medical observation of the patient.

5. The method of claim 4, wherein continuously monitoring over time the Doppler flow signal and the at least one characteristic during the surgery, medical procedure, or medical observation of the patient comprises:
collecting, by the processor of the blood flow monitor, a running average, or a running mean, of abnormal values of the at least one characteristic during the surgery, medical procedure, or medical observation of the patient.

6. The method of claim 1, wherein the at least one characteristic associated with the targeted organ blood flow of the patient from the Doppler flow signal comprises:
a flow rate of the targeted organ blood flow of the patient; and/or
comprises a blood flow index of the targeted organ blood flow of the patient; and/or
an autoregulation profile of the targeted organ blood flow of the patient.

7. The method of claim 6, wherein the at least one characteristic associated with the targeted organ blood flow of the patient from the Doppler flow signal comprises a blood flow index of the targeted organ blood flow of the patient, the targeted organ blood flow is a renal blood flow of the patient, and the blood flow index comprises a Venous Impedance Index (VII) and/or a Renal Resistive Index (RRI).

8. The method of claim 6, wherein the at least one characteristic associated with the targeted organ blood flow of the patient from the Doppler flow signal comprises an autoregulation profile of the targeted organ blood flow of the patient, and renal blood flow of the patient is the targeted organ blood flow, and the method further comprises:
obtaining an arterial pressure of the patient with a pressure sensor attached to the patient;
estimating by the processor of the blood flow monitor a flow rate of the renal blood flow of the patient from the Doppler flow signal of the renal blood flow; and
monitoring by the processor changes in the flow rate of the renal blood flow over time;
monitoring by the processor changes in the arterial pressure over time;
evaluating a correlation or non-correlation by the processor between the changes in the arterial pressure and the changes in the flow rate of the renal blood flow; and
determining by the processor the autoregulation profile of the renal blood flow of the patient based on the correlation or non-correlation between the changes in the arterial pressure and the changes in the flow rate of the renal blood flow.

9. The method of claim 8, wherein the pressure sensor is:
attached to the patient by a radial arterial catheter; or
attached to the patient by a femoral arterial catheter; or
attached non-invasively to an extremity of the patient.

10. The method of claim 1, further comprising:
estimating, by the processor of the blood flow monitor, a real-time organ injury risk score of the patient from the at least one characteristic and the baseline value of the at least one characteristic; and
outputting in real time to the display a representation of the real-time organ injury risk score of the patient over time.

11. The method of claim 10, further comprising:
tracking over time, by the processor, the real-time organ injury risk score of the patient to determine a final organ injury risk score of the patient.

12. The method of claim 1, further comprising:
executing beamformer software code by the processor to track-scan the Doppler flow signal of the targeted organ blood flow of the patient with a two-dimensional phased array of transducer elements of the ultrasound transducer probe to continuously sense the Doppler flow signal of the targeted organ blood flow of the patient during the surgery, medical procedure, or medical observation without an ultrasound operator.

13. The method of claim 12, further comprising:
executing the beamformer software code by the processor to emit a set of sequential beams from the array of transducer elements into the abdomen of the patient to track a center of the targeted organ blood flow relative to the array of transducer elements;
focusing, by the processor and the beamformer software code, each beam from the set of sequential beams in different locations; and
adjusting, by the processor and the beamformer software code, the position of the set of sequential beams onto the center of the targeted organ blood flow to maintain the Doppler flow signal of the targeted organ blood flow of the patient.

14. A system comprising:
an ultrasound transducer probe comprising a two-dimensional array of transducer elements configured to measure a Doppler flow signal of a targeted organ blood flow of a patient;
an adhesive patch connected to the ultrasound transducer probe and configured to attach the ultrasound transducer probe to the patient and maintain contact between the patient and the ultrasound transducer probe without an operator;
a blood flow monitor in communication with the ultrasound transducer probe, wherein the blood flow monitor comprises:
a system memory that stores monitoring software code; and
a processor configured to execute the monitoring software code to:
determine at least one characteristic associated with the targeted organ blood flow of the patient; and
monitor over time the at least one characteristic associated with the targeted organ blood flow of the patient during a surgery, medical procedure, or medical observation of the patient.

15. The system of claim 14, further comprising:
a display in communication with the processor to receive and show a continuous reading of the Doppler flow signal from the ultrasound transducer probe and a representation of the at least one characteristic over time.

16. The system of claim 14, wherein the processor is configured to execute the monitoring software code to:
determine a baseline value of the at least one characteristic associated with the targeted organ blood flow of the patient.

17. The system of claim 16, wherein the at least one characteristic associated with the targeted organ blood flow of the patient from the Doppler flow signal comprises:
a flow rate of the targeted organ blood flow of the patient; and/or
a blood flow index of the targeted organ blood flow of the patient; and/or
an autoregulation profile of the targeted organ blood flow of the patient.

18. The system of claim 17, wherein the at least one characteristic associated with the targeted organ blood flow of the patient from the Doppler flow signal comprises a blood flow index of the targeted organ blood flow of the patient, the targeted organ blood flow is a renal blood flow of the patient, and the blood flow index comprises a Venous Impedance Index (VII) and/or a Renal Resistive Index (RRI).

19. The system of claim 17, wherein the at least one characteristic associated with the targeted organ blood flow of the patient from the Doppler flow signal comprises an autoregulation profile of the targeted organ blood flow of the patient, and the processor is configured to execute the monitoring software code to:
collect a running sum of a duration of inactive autoregulation of the targeted organ blood flow of the patient during the surgery, the medical procedure, or the medical observation.

20. The system of claim 19, wherein the system further comprises:
a hemodynamic pressure sensor configured to measure an arterial pressure of the patient,
wherein the processor is configured to monitor the arterial pressure of the patient measured by the hemodynamic pressure sensor, and
wherein the processor is configured to determine the autoregulation profile of the targeted organ blood flow of the patient based on changes in the targeted organ blood flow determined from the Doppler flow signal over time and based on changes in the arterial pressure of the patient over time measured by the hemodynamic pressure sensor.

21. The system of claim 20, wherein the hemodynamic pressure sensor is:
connected to a radial arterial catheter; or
connected to a femoral arterial catheter; or
a non-invasive hemodynamic pressure sensor configured for attachment to an extremity of the patient.

22. The system of claim 14, wherein the monitoring software code comprises organ injury monitoring software code, and wherein the processor is configured to execute the organ injury monitoring software code to:
estimate a real-time organ injury risk score of the patient from the at least one characteristic; and
output in real time to the display a representation of the real-time organ injury risk score of the patient over time.

23. The system of claim 22, wherein the processor is configured to execute the organ injury monitoring software code to:
track over time the real-time organ injury risk score of the patient to determine a final organ injury risk score of the patient.

24. The system of claim 14, wherein the two-dimensional array of transducer elements of the ultrasound transducer probe comprises a phased array of transducer elements.

25. The system of claim 24, wherein the system memory stores probe control software code with beamformer software code, and wherein the processor is configured to execute the beamformer software code to:
to track-scan the Doppler flow signal of the targeted organ blood flow of the patient by emitting multiple ultrasound beams from the phased array of transducer elements to track the Doppler flow signal of the targeted organ blood flow of the patient relative to the phased array of transducer elements.
